(19) United States
(12) Patent Application Publication
(10) Pub. No.: US 2011/0046704 A1
(43) Pub. Date: Feb. 24, 2011

(54) INFLATABLE THERMAL NEONATAL BLANKET

(76) Inventors: Arthur Everardus Officier, Epe (NL); Berend Jan Teunissen, Haaksbergen (NL)

Correspondence Address: KNOX PATENTS, P.O. BOX 30034, KNOXVILLE, TN, 37930-0034, US

(21) Appl. No.: 12/544,855

(22) Filed: Aug. 20, 2009

(51) Int. Cl. A61F 7/00

(52) U.S. Cl. 607/104

(57) ABSTRACT

The invention relates to an inflatable thermal blanket for providing conditioned air to a neonatal human or neonatal animal body, said thermal blanket comprising:

a base sheet of an air impermeable material, in use remote from said body;

a top sheet of an air permeable material, in use in contact with said body;

a peripheral connection joining said base sheet and top sheet to define an air chamber;

multiple connections distributed within said peripheral connection across interior surface regions of said base sheet and top sheet and connecting said base sheet and top sheet,

an inlet port for providing the conditioned gas to said inflatable chamber,

said peripheral connection having a length of about 70-100 cm and a width of about 50-70 cm, and

said multiple connections defining a central part and outer channels along the longitudinal ends of the thermal blanket, said central part having a length of about 30-60 cm and a width of about 20-40 cm.

### BACKGROUND

The present invention relates to a blanket used to covering at least a portion of the body of a neonatal human, or other animal, in order to bath the body portion in a conditioned gas. More specifically, the present invention is related to an inflatable thermal blanket for providing a conditioned air to the body of a user.

Inflatable thermal blankets are commonly used as so called upper body blankets. They are positioned on top of or over a body or portion of a body. These blankets are often used in hypothermia situations, pre-, peri- or post-operative. In these thermal blankets, warm air is inserted from a hyperthermia unit, though a supply hose, into the inlet port of the blanket. After inflating the blanket, this warm air is communicated though a distribution means in the blanket to a body or portion of a body. Usually, an air permeable layer is used for releasing the warm air to the body.

These known blankets are placed over a person in order to keep them warm. During surgery or other medical treatments when access to various parts of the body is needed, there is need for a device for keeping a body conditioned.

It was found that in particular for neonatal humans, i.e. neonatal babies, current thermal blankets and other known means for conditioning a patient, for instance the temperature, did not satisfy.

### SUMMARY OF THE INVENTION

The invention aims to provide a new type of thermal blankets.

In particular, the inventions seek to provide a device and method for conditioning a body of a neonatal, in particular a neonatal baby.

In particular, the invention seeks to provide a device and method of conditioning a body of a neonatal baby while allowing access to the body.

According to a first aspect of the invention this is realized with an inflatable thermal blanket for providing conditioned air to a neonatal human or neonatal animal body, said thermal blanket comprising:

a base sheet of an air impermeable material, in use remote from said body;

a top sheet of an air permeable material, in use in contact with said body;

a peripheral connection joining said base sheet and top sheet to define an air chamber;

multiple connections distributed within said peripheral connection across interior surface regions of said base sheet and top sheet and connecting said base sheet and top sheet,

an inlet port for providing the conditioned gas to said inflatable chamber,

said peripheral connection having a length of about 70-100 cm and a width of about 50-70 cm, and

said multiple connections defining a central part and outer channels along the longitudinal ends of the thermal blanket, said central part having a length of about 30-60 cm and a width of about 20-40 cm.

The invention furthermore provides an inflatable thermal blanket for providing conditioned air to a neonatal human or neonatal animal body positioned under said thermal blanket, said thermal blanket comprising:

a base sheet of an air impermeable material, in use remote from said body;

a top sheet of an air permeable material, in use in contact with said body;

a peripheral connection joining said base sheet and top sheet to define an air chamber;

multiple connections distributed within said peripheral connection across interior surface regions of said base sheet and top sheet and connecting said base sheet and top sheet,

an inlet port for providing the conditioned gas to said inflatable chamber,

said peripheral connection having a length of about 70-100 cm and a width of about 50-70 cm, and

said multiple connections defining a central part and outer channels along the longitudinal ends of the thermal blanket, said outer channels having an uninflated width of about 10-40 cm defining, in use, an outer channel having a height about at least 1.5 times heigher than the central part.

The invention furthermore provides an inflatable thermal blanket for providing conditioned air to a neonatal human or neonatal animal body positioned on top of said thermal blanket, said thermal blanket comprising:

a base sheet of an air impermeable material, in use remote from said body;

a top sheet of an air permeable material, in use in contact with said body;

a peripheral connection joining said base sheet and top sheet to define an air chamber;

multiple connections distributed within said peripheral connection across interior surface regions of said base sheet and top sheet and connecting said base sheet and top sheet,

an inlet port for providing the conditioned gas to said inflatable chamber,

said peripheral connection having a length of about 70-100 cm and a width of about 45-70 cm, and

said multiple connections defining a central part and outer channels along the longitudinal ends of the thermal blanket, outer channels having a width of about 10-20 cm.

The invention furthermore provides a method for conditioning a neonatal person, in particular for conditioning the temperature of a neonatal person, said method comprising the steps of

providing a blanket on a surface, said blanket comprising:
a base sheet of an air impermeable material, in use remote from said neonatal person;
a top sheet of an air permeable material, in use in contact with said neonatal person;
a peripheral connection joining said base sheet and top sheet to define an air chamber;
multiple connections distributed within said peripheral connection across interior surface regions of said base sheet and top sheet and connecting said base sheet and top sheet, said multiple connections defining an inflatable central part and inflatable outer channels along the longitudinal ends of the thermal blanket and

an inlet port for providing the conditioned gas to said inflatable central part and inflatable outer channels, and

positioning the neonatal person on top of said blanket on said central part of said blanket.

The invention furthermore provides a method for conditioning a neonatal person, in particular for conditioning the temperature of a neonatal person, said method comprising the steps of

positioning a neonatal person on a surface;

providing a inflatable thermal blanket, said blanket comprising:
a base sheet of an air impermeable material, in use remote from said neonatal person;
a top sheet of an air permeable material, in use in contact with said neonatal person;
a peripheral connection joining said base sheet and top sheet to define an air chamber;
multiple connections distributed within said peripheral connection across interior surface regions of said base sheet and top sheet and connecting said base sheet and top sheet, said multiple connections defining an inflatable central part and inflatable outer channels along the longitudinal ends of the thermal blanket and

an inlet port for providing the conditioned gas to said inflatable central part and inflatable outer channels, and

positioning said blanket on top of the neonatal person with said central part of said blanket largerly on said neonatal person.

The present invention provides an inflatable thermal blanket capable of providing a gas, such as for example heated air, to for instance the underbody of a neonatal user. The thermal blanket includes an inflatable portion for receiving the conditioned gas under pressure and for being positioned under the body of a user. In other words, a body is positioned on top of the thermal blanket. The inflatable portion is defined by an air impermeable base sheet and an air permeable top sheet which is being directed to the body. In an embodiment, it may comprise one or more air permeable barrier sheets positioned between the base sheet and top sheet. The inflatable portion also includes an inlet port, being connected to a source of conditioned gas.

In another embodiment, the thermal blanket is provided over the body, i.e. largely covering the body. In this use, the air permeable sheet is in contact with the body. This thermal blanket and method of the invention provides the possibility of keeping a neonatal person or animal warm, for instance during medical treatment. The blanket of the invention is for instance used during surgery. Clinical studies showed that using the blanket of the invention provides the most effective way of keeping a body warm. In fact, it has the highest efficiency in comparison to other methods. Additionally, the blanket of the invention is disposable, in contrast to other means for warming a body or keeping a body warm. This makes the use in medical environment in particular easy.

In an embodiment, the top sheet has an air permeability of 1-400 mm/sec measured at a pressure drop of 100 Pa. This, the diffusion of air is further optimized in this type of blanket.

In an embodiment, the inlet port is positioned symmetrically compared to the longitudinal axis of the blanket.

In an embodiment, the inlet port is connected to a peripherical dual inlet channel, to in use split the flow to either side of the blanket. Said inlet channel is equipped with an opening, preferably positioned ahead of the inlet port, to allow a part of the airflow to be directed straight to the centre section.

In an embodiment, the splitted flow is dispersed to an outer channel and a central part, covering the body. In an embodiment, the outer channels and the central part comprise open contacts at both the upstream and downstream side of the outer channels. Thus, conditioned gas can be diffused and is allowed to exit over the entire area of the blanket.

In an embodiment, the central part comprises multiple seal connections between the base sheet, barrier sheet(s) and top sheet such as dots, strips, channels etc. Thus, the layers of the blanket are connected to one another.

In an embodiment, the base and/or top sheets extend outward beyond said peripheral connection.

In an embodiment the inlet port is connected to a dual inlet channel, creating a splitted flow to either side of the body.

In an embodiment multiple connections are provided for allowing the splitted flow to be dispersed to at least one of said outer channels, and said central part of said blanket.

In an embodiment the outer channels and the central part are in air contact with one another at both the upstream and downstream side of the outer channels.

In an embodiment the central part comprises multiple seal connections between the base sheet and top sheet, said multiple seals formed as dots, strips and/or channels.

In an embodiment the outer channels have a width of about 12-18 cm. In a more particular embodiment, the outer channels have a width of about 13-17 cm. The specific sizes are adapted to provide a better air flow for neonatals.

In an embodiment the blanket has a length of about 70-120 cm and a width of about 45-90 cm.

In an embodiment the peripheral connection having a length of about 75-90 cm and a width of about 50-65 cm.

In an embodiment the inflatable blanket is further provided with multiple connections within said central portion, limiting in use, when filled with conditioned air, the height of said central portion to about 5-15 cm while in use the height of said outer channels is about 10-20 cm. In an embodiment, these dimensions are selected in such a way that the height of the outer channels is larger than the height of the central part. The advantage of this is clarified in the description of an embodiment.

In an embodiment, the outer channels have an uninflated width of about 10-40 cm defining, in use, an outer channel having a height about at least 1.5 times heigher than the central part. This provides air covering the neonatal like a cushion of conditioned air. In an embodiment, the outer channel in use, i.e. in inflatated condition, can be at least 2 times as high as the central part.

In an embodiment, the base sheet comprises a heat reflective layer. To that end, for instance an aluminium coating layer can be provided, or another heat reflective layer which is known from other fields of art.

Several aspects of the claims or features of the depending claims or the description can be combined in order to provided additional advantages. Furthermore, features of the description and/or the depending claims may form the basis of one or more divisional patents.

### DESCRIPTION OF THE DRAWINGS

The invention will be further elucidated referring to an embodiment of a thermal blanket and its use shown in the attached drawings, showing in:

FIG. 1 a top view of a small version of an inflatable thermal blanket of the present invention;

FIG. 2 a projection of a neonatal human body at the inflatable thermal blanket of the present invention

FIG. 3 a projection of a neonatal human body under the inflatable thermal blanket

FIG. 4a a cross section of a part of the blanket as indicated in FIG. 3;

FIG. 4b a cross section of a part of the blanket of FIG. 3, and

FIG. 5 a schematic sideview, in longitudinal direction of the thermal blanket of FIG. 2 in use.

### DETAILED DESCRIPTION OF EMBODIMENTS

In the drawings, similar features have the same reference numbers.

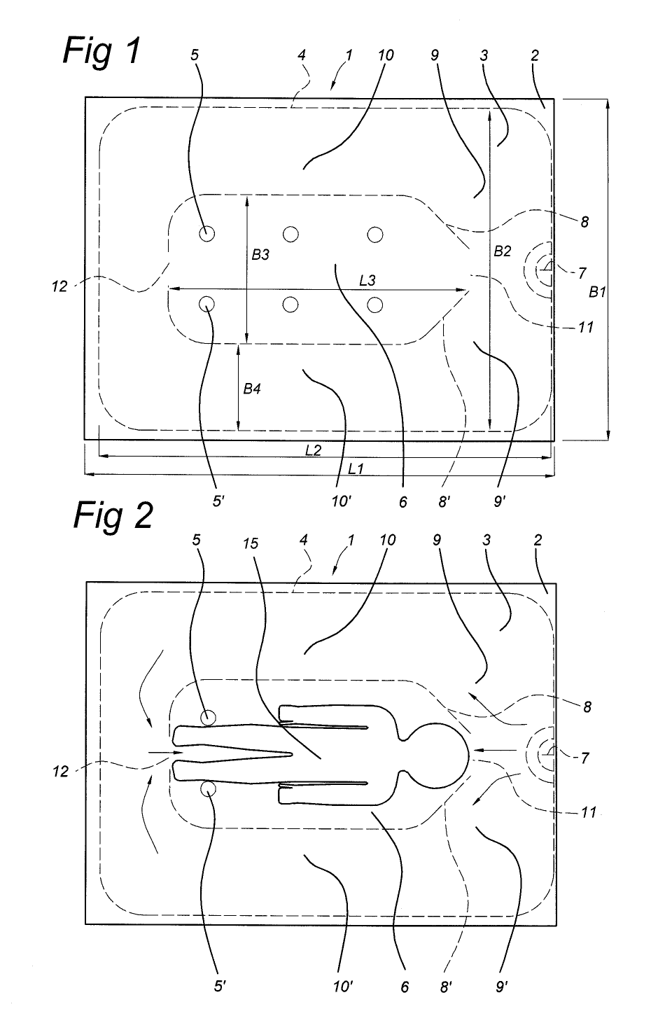

An embodiment of the thermal blanket of the present invention is illustrated generally in FIG. 1 in top view.

Thermal blanket 1 of FIG. 1 has a base sheet 2. This base sheet 2 is of an air impermeable material. Attached to the base sheet 2 is a top sheet 3. These sheets 2, 3 are attached to one another via a peripheral connection 4. This peripheral connection 4 is indicated with a dotted line. Thus, base sheet 2 and top sheet 3 attached to one another via peripheral connection 4 form an inflatable air chamber or inflatable portion. The inflatable portion has an inlet port 7. In FIG. 1, thermal blanket 1 is positioned with the base sheet 2 resting on a flat surface.

FIG. 2 depicts a projected view of a neonatal baby 15, lying on top of blanket 1 of FIG. 1. This view clearly direct the position of the head, arms, legs and thorax of a neonatal person 15.

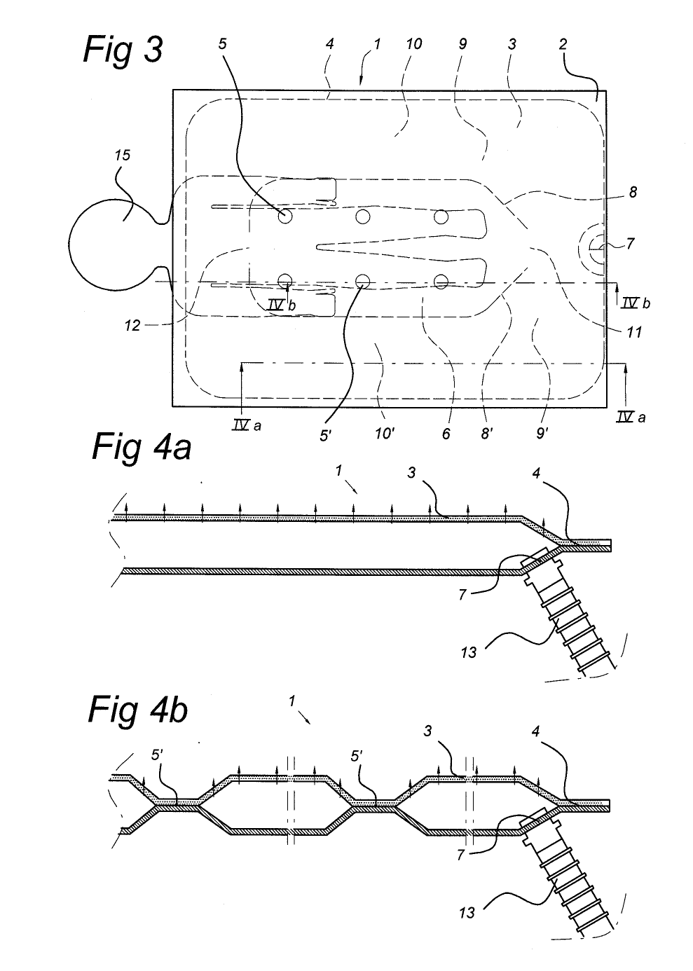

FIG. 3 depicts a projected view of a neonatal human body 15, lying below the blanket 1 of FIG. 1, i.e. the blanket 1 is positioned over a large part of the neonatal 15.

The thermal blanket 1 includes an inflatable portion for receiving the conditioned gas under pressure and for being positioned under or over the body of a user. The inflatable portion is defined by the air impermeable base sheet 2 the air permeable top sheet 3. In use as a so called underbody blanket 1, this top sheet 3 is directed towards the body. resting on top of the blanket. The base sheet 2 rests on a supporting surface, for instance a bed or operating or surgical table. In use as a so called upperbody blanket, this top sheet 3 is directed towards the body resting below the blanket. The body is subsequently resting on a supporting surface, for instance a bed or a surgical table.

Furthermore, the inflatable portion is bounded, circumferentially, by the peripheral connection 4. The inflatable portion also includes an inlet port 7, in use connected to a source of conditioned gas. The inlet port 7 is in an embodiment positioned symmetrically with respect to the longitudinal axis of the blanket as depicted at FIG. 2.

In use, the conditioned gas enters the blanket 1 via the inlet port 7 between the base sheet 2 and the top sheet 3.

In addition to the peripheral connection 4, the top sheet 3 and base sheet 2 can be connected via seals 8, 8′. These seal lines 8, 8′ are usually continuous seal lines connecting the air permeable top sheet 3 and air impermeable base sheet 2. These seals 8, 8′ define, in this embodiment, outer channels 10, 10′, and a central part 6. The seals 8, 8′ define a passage 12 at one end of the blanket opposite air inlet 7. The seals further define a further passage 11 at the opposite end of the blanket, the side of air inlet 7. Thus, in use air can enter the central part 6. The seals 8, 8′ further have angled parts near the air inlet 7, thus in use splitting an air flow from air inlet 7 in three parts, two parts flowing through outer channels 10, 10′, and one part flowing into the central part 6. The angled parts of seals 8, 8′ thus form inlet channels 9, 9′. The thus defined outer channels 9, 9′ and the central part 6 are mutually in fluid connection, and are in fluid connection with air inlet 7.

After entering the blanket 1, the conditioned gas is directed to a dual inlet channel 9, 9′ configuration, creating a splitted flow to either side of the body on top of the blanket. Flow splitting is directed by a symmetrical positioned angled seals 8, 8′. After passing the angled seals 8, 8′, the splitted flow from the inlet channels 9, 9′ is dispersed to two peripherical outer channels 10, 10′ and a central part 6, covered by the body lying on the blanket 1 or under the blanket 1.

The central part 6 in an embodiment has, within its boundarie formed in this embodiment by seals 8, 8′, multiple seal connections 5 connecting the base sheet 2 and top sheet 3 together. These seal connections can be dots, strips, channels, etc. The seal connections are provided to distribute the flow of air and to control the height of the central part 6 of the inflated blanket 1. In fact, the current blanket can use the type of connections and pattern of connections as discussed in U.S. Ser. No. 12/342,933. This application is referred to as if fully set forth in this description. Thus, the seals 8, 8′ and multiple seal connections 5, 5′ in the embodiment provide multiple connections distributed within the peripheral connection.

The conditioned gas leaves the inflated blanket 1 through the top layer 3 to the body via multiple exhaust ports of any shape or via any porous material, for instance meltblown material. In an embodiment, the air permeable sheet 3 comprises a meltblown material. Meltblown material or similar porous material allows a very even spreading of discharged, conditioned air. Specific materials suited for this purpose are known to a skilled person in this field.

The flow in the outer channels 10, 10′ is directly contacting the central part 6 by the presence of flow connections or on or more passages at the upstream side 11 just ahead of the inlet port at the beginning of the outer channels and one or more passages at the downstream side 12 at the end of the outer channels 10, 10′, which outer channels 10, 10′ are positioned along the longitudinal axis.

When in use as underbody blanket, conditioned air flowing out through the air permeable sheet 3 at the upstream side of the central part will warm the head, neck and thorax sides; conditioned air flowing out through the air permeable sheet 3 at the downstream side of the central part will warm the legs and lower part of the thorax; flow at the central part will warm the thorax and arms. When using a nonwoven fibrous material, which as such is well known in the art, the flow out air will be gently and almost form a cushion or conditioned air between the blanket 1 and the neonatal 15.

In an embodiment, the outer dimensions of the base and/or top sheets may be larger than the peripheral connection dimensions in order to fix the blanket to the body by fixation provisions at the peripheral longitudinal sides whilst not affecting the shape of the outer channels.

In an embodiment, the base sheet 2 comprises a heat reflective layer. A heat reflective layer was found to increase effectiveness of the blanket, in particular with respect to its thermal performance.

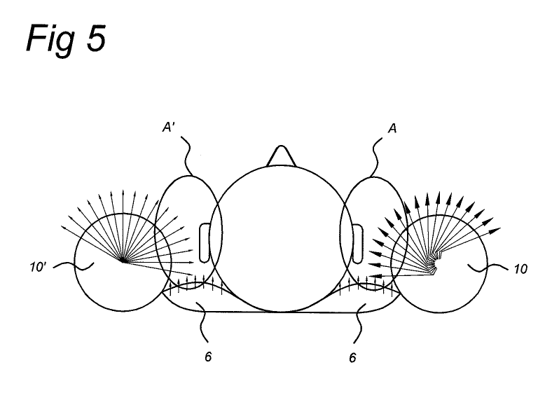

In FIGS. 4a and 4b, cross sections through part of the thermal blanket in use is shown, as indicated in FIG. 3. Via a tube or hose 13, conditioned air is provided to inlet port 7. This conditioned air fills the air chamber formed by the air permeable top sheet 3, and air impermeable base sheet 2. The air chamber is further bounded by the peripheral connection 4 which holds together the different layers forming the thermal blanket 1. In the cross sections, the thickness of the central part (FIG. 4b) and of the outer channels (FIG. 4a) seems equal. In use, the positioning of the multiple seal connections 5 in the central part 6 and of the distance between seals 8, 8′ and peripheral connection 4 provides that in use the outer channels 10, 10′ are thicker than the central part 6. This is demonstrated in FIG. 5 schematically. The neonatal 15 is surrounded by a thicker band formed by at least the outer channels 10, 10′. In this way, in use a cushion of encompassed conditioned air can be spread over the neonatal. The seal connections 5 in the central part, together with the weight of the neonatal, reduces the thickness of the central part 6. FIG. 5 does not show the thicker part of both ends of the blanket 1, i.e. the parts near openings or passages 12 and 11. Thus, in use the neonatal 15 will be surrounded by a relatively higher/thicker band or tyre from which conditioned air flows over the neonatal 15, positioned on top of the blanket 1 in the central part 6, as a sort of cushion of conditioned air.

In view of FIG. 5, in an embodiment the outer edge of outer channels 10, 10′, i.e., those parts which evaporate or blow air away from the neonatal 15, may be impermeable. This can for instance be accomplished by locally adding a strip of an air impermeable layer over the air permeable layer. Alternatively, the air impermeable base sheet 2 can be wider than the air permeable top sheet 3. Its longitudinal sides can be folded to the air permeable base sheet 2 and attached to it. This may be easy to produce. In those ways, the outer channels 10, 10′ only blow air for instance over the radial part directed towards the neonatal 15. This will result in a flow in region A, A′, and, if the flow is stronger, over the neonatal 15, thus covering the neonatal 15 in a cushion of conditioned air. This feature may reduce the amount of conditioned air needed. Furthermore, treating medical staff, for instance, will not be subjected to the flow of conditioned air.

It was found that if the peripheral connection 4 of the blanket 1 has a length L2 of about 70-100 cm and a width B2 of about 50-70 cm, it is capable of being used for a neonatal in almost any situation. If it has these dimensions, it can be used for both resting a neonatal on top of the blanket 1 as shown in FIG. 2, or for covering most part of the neonatal, as shown in FIG. 3. In fact, the blanket intended for sale usually has a length L1 of about 85 cm and a width B1 of about 61 cm. The impermeable base sheet 2 or permeable sheet 3 can have larger dimensions.

It was found that if the central part 6 has a length L3 of about 30-60 cm and a width B3 of about 20-40 cm, it it provised a configuration which in use allows a blanket of conditioned air to be formed over the neonatal if it is provided on top of the blanket 1. Furthermore, it allows proper positioning and it comforts the neonatal 15. In fact, providing the dimensions of the outer channels 10, 10′ and the central part 6 with seal connections 5, 5′ and their mutual distances according to the current design was found to strengthen this effect.

The distance in width between the seals 8, 8′ and the seal connections 5, 5′, respectively, will be about 4-10 cm. In an embodiment, the distance between seal connections mutually will be about 15-12 cm. This distance provides a proper blanket thickness when filled with air.

In an embodiment, the distance between the peripheral connection 4 and the seals 8, 8′ will be about at least 10 cm. In an embodiment, it can be less than 20 cm. In an embodiment, it is about 12-18 cm. It was found that such a width of the outer channels provides a band or tyre around the central part 6 which is beneficial for the neonatal 15.

The top sheet and base sheet can be adhered to one another using heat sealing or another know sealing process or method, such as glueing.

In use, a neonatal person is positioned either on the blanket as shown in FIG. 2 (and FIG. 5) or under the blanket as shown in FIG. 3. It is also possible to provise a blanket on a surface, place a neonatal 15 on top of it as shown in FIG. 2, and subsequently provide a similar blanket 1 over the neonatal in the way shown in FIG. 3. In this way, the neonatal is surrounded by a flow of conditioned air.

It will also be clear that the above description and drawings are included to illustrate some embodiments of the invention, and not to limit the scope of protection. Starting from this disclosure, many more embodiments will be evident to a skilled person which are within the scope of protection and the essence of this invention and which are obvious combinations of prior art techniques and the disclosure of this patent.

1-18. (canceled)

19. An inflatable thermal blanket for providing a conditioned gas to a neonatal body, said inflatable thermal blanket comprising:
a base sheet composed of an air impermeable material and adapted for use remote from the neonatal body;
a top sheet composed of an air permeable material and adapted for use in contact with the neonatal body;
a peripheral connection joining said base sheet and the top sheet at a periphery of said inflatable thermal blanket to define an inflatable air chamber, said peripheral connection having a length of about 70-100 cm and a width of about 50-70 cm;
a plurality of inner connections distributed within said peripheral connection across interior surface regions of said base sheet and said top sheet and connecting said base sheet and said top sheet at an interior of the inflatable thermal blanket, said plurality of connections defining a central part and outer channels along the longitudinal ends of the thermal blanket, said central part having a length of about 30-60 cm and a width of about 20-40 cm; and
an inlet port adapted to provide the conditioned gas to said inflatable air chamber.

20. The inflatable thermal blanket of claim 19, wherein said top sheet has an air permeability of 1-400 mm/sec measured at a pressure drop of 100 Pa.

21. The inflatable thermal blanket of claim 19, wherein said inlet port is positioned symmetrically compared to the longitudinal axis of the blanket.

22. The inflatable thermal blanket of claim 19, further comprising a dual inlet channel,
wherein said inlet port is connected to the dual inlet channel to thereby create a bifurcated flow of air to either side of the neonatal body.

23. The inflatable thermal blanket of claim 22, wherein said multiple connections are provided for allowing the bifurcated flow of air to be dispersed to at least one of said outer channels and said central part of said blanket.

24. The inflatable thermal blanket of claim 23, wherein the outer channels and the central part are in air contact with one another at both an upstream and a downstream side of said outer channels.

25. The inflatable thermal blanket of claim 23, wherein said central part comprises is defined by said plurality of inner connections.

26. The inflatable thermal blanket of claim 19, wherein one of said base sheet and said top sheet extends outwardly beyond said peripheral connection.

27. The inflatable thermal blanket of claim 19, wherein said base sheet comprises a heat reflective layer.

28. An inflatable thermal blanket for providing a conditioned gas to a neonatal body positioned under a lower surface thereof, said thermal blanket comprising:
a base sheet composed of an air impermeable material and adapted for use remote from the neonatal body;
a top sheet composed of an air permeable material and adapted for use in contact with the neonatal body;
a peripheral connection joining said base sheet and top sheet at a periphery thereof to define an inflatable air chamber, said peripheral connection having a length of about 70-100 cm and a width of about 50-70 cm;
a plurality of inner connections distributed within said peripheral connection across interior surface regions of said base sheet and said top sheet and connecting said base sheet and said top sheet; and
an inlet port adapted to provide the conditioned gas to said inflatable air chamber,
wherein said plurality of connections define a central part and outer channels along the longitudinal ends of the inflatable thermal blanket, said outer channels having an uninflated width of about 10-40 cm defining, in use, an outer channel having a height about at least 1.5 times greater than the height at said central part.

29. An inflatable thermal blanket for providing a conditioned gas to a neonatal body positioned on an upper surface thereof, said thermal blanket comprising:
a base sheet composed of an air impermeable material and adapted for use remote from the neonatal body;
a top sheet composed of an air permeable material and adapted for use in contact with the neonatal body;
a peripheral connection joining said base sheet and said top sheet to define an air chamber, said peripheral connection having a length of about 70-100 cm and a width of about 45-70 cm;
a plurality of connections distributed within said peripheral connection across interior surface regions of said base sheet and said top sheet and connecting said base sheet and said top sheet; and
an inlet port for providing the conditioned gas to said inflatable chamber,
wherein said plurality of connections define a central part and outer channels along the longitudinal ends of the inflatable thermal blanket, said outer channels having a width of about 10-20 cm.

30. The inflatable thermal blanket of claim 29, wherein said outer channels have a width of about 12-18 cm.

31. The inflatable thermal blanket of claim 29, wherein said outer channels have a width of about 13-17 cm.

32. The inflatable thermal blanket of claim 29, wherein said inflatable thermal blanket has a length of about 70-120 cm and a width of about 45-90 cm.

33. The inflatable thermal blanket of claim 29, wherein said peripheral connection has a length of about 75-90 cm and a width of about 50-65 cm.

34. The inflatable thermal blanket of claim 29, further comprising a second plurality of connections provided within said central portion,

35. The inflatable thermal blanket of claim 34, wherein when filled with the conditioned gas, the height of said central portion is about 5-15 cm and the height of said outer channels is about 10-20 cm.

36. A method for conditioning the temperature of a neonatal body, said method comprising:
providing a thermal blanket on a surface, said thermal blanket comprising:
a base sheet composed of an air impermeable material and adapted for use remote from the neonatal body;
a top sheet composed of an air permeable material and adapted for use in contact with the neonatal body;
a peripheral connection joining said base sheet and top sheet to define an air chamber;
a plurality of connections distributed within said peripheral connection across interior surface regions of said base sheet and said top sheet and connecting said base sheet and said top sheet, said plurality of connections defining an inflatable central part and inflatable outer channels along the longitudinal ends of the thermal blanket; and
an inlet port adapted to provide a conditioned gas to said inflatable central part and said inflatable outer channels, and then
positioning the neonatal body on an upper surface of said thermal blanket at said inflatable central part thereof.

37. A method for conditioning the temperature of a neonatal body, said method comprising:
positioning the neonatal body on a surface;
providing an thermal blanket comprising:
a base sheet composed of an air impermeable material and adapted for use remote from the neonatal body;
a top sheet composed of an air permeable material and adapted for use in contact with the neonatal person;
a peripheral connection joining said base sheet and said top sheet to define an air chamber;
a plurality of connections distributed within said peripheral connection across interior surface regions of said base sheet and said top sheet and connecting said base sheet and said top sheet, said plurality of connections defining an inflatable central part and inflatable outer channels along the longitudinal ends of the thermal blanket; and
an inlet port adapted to provide a conditioned gas to said inflatable central part and said inflatable outer channels, and then
positioning said thermal blanket on the neonatal body such that said inflatable central part contacts the neonatal body.
